(19) United States
(12) Patent Application Publication
(10) Pub. No.: US 2018/0042912 A1
(43) Pub. Date: Feb. 15, 2018

(54) MEDICAL PRODUCT AND METHOD FOR ELIMINATING SYMPTOMS OF NICOTINE WITHDRAWAL

(71) Applicants: Nicholas A. Havercroft, Scunthorpe (GB); Donald M. Pell, St. Petersburg, FL (US); Michael P. Spuza, St. Petersburg, FL (US); Govindan P. Nair, St. Petersburg, FL (US)

(72) Inventors: Nicholas A. Havercroft, Scunthorpe (GB); Donald M. Pell, St. Petersburg, FL (US); Michael P. Spuza, St. Petersburg, FL (US); Govindan P. Nair, St. Petersburg, FL (US)

(21) Appl. No.: 15/234,035

(22) Filed: Aug. 11, 2016

(51) Int. Cl. A61K 31/465; A61K 9/48; A61K 47/12; A61K 47/02; A61K 47/10

(52) U.S. Cl. CPC A61K 31/465; A61K 47/02; A61K 47/10; A61K 47/12; A61K 9/485; A61K 9/4858

(57) ABSTRACT

In essence, the present invention relates to a medical product including between about 94% and about 98% of a saline solution, preferably a normal saline solution containing about 0.9% sodium chloride dissolved in water, about ⅛^{th} of 1 milligram of relatively pure nicotine and about 3 milligrams of relatively pure nicotine. The medical product also includes about 1% and 3% of one or more flavors selected from the group consisting of citrus, tobacco, apple, cherry, maple, menthol, hazelnut, peach, lemon, vanilla and chocolate and 1% citric acid for taste. In addition, the medical product includes about 1% to 2% of ethyl alcohol for cleaning a micron mesh grid that generates particles of pure nicotine of from 2 to 5 microns that are drawn through an entrainment port, through a capsule containing the medical product through the micron mesh grid and into the individual's lungs and into their bloodstream.

### FIELD OF THE INVENTION

This invention relates to a medical product and method for eliminating the symptoms of nicotine withdrawal for those addicted to nicotine as a result of the use of tobacco products.

### BACKGROUND FOR THE INVENTION

It is common for former smokers to state that quitting smoking was the hardest thing that they have ever done. An observation of a physician who worked in an addiction center is that most addicts continue to smoke, commenting that overcoming drug addiction was easy compared to quitting smoking. Furthermore, smoking was not illegal, and was relatively cheaper than drugs.

Within about two hours of smoking a cigarette the human body craves another, and with increased frequency the craving becomes stronger. Cigarettes provide almost instant gratification.

In today's market there have been a number of approaches to overcome an addiction to nicotine. Many nicotine replacement products have been unsatisfactory due to delivery of low levels of nicotine. However, one approach as disclosed in our earlier U.S. patent application Ser. No. 15/174,178 filed on Jun. 6, 2016 and entitled Pulmonary Device, Method and Systems for Delivering a Pharmaceutical Product to An Individual has been effective to eliminate the symptoms of withdrawal from a nicotine containing product.

As disclosed in our aforementioned patent application it has now been recognized that inhaling very small amounts of 2 to 5 micron particles of a nicotine saline solution will avoid withdrawal symptoms. Nevertheless, it is important to prevent the inhaler from delivering an excess amount of nicotine particularly for young persons and the potential death of pets due to the ingestion of one or more capsules. Accordingly, an inhaler for limiting the time of an initial inspiration and to prevent second or subsequent inspirations for a preselected time following an initial or previous inspiration is provided. It is also important to limit the inhaler to use replaceable cartridges as opposed to refillable containers to avoid having relatively untrained individuals working with toxic materials and exposing the nicotine solution to contamination, bacteria, fungus and yeast.

Notwithstanding the above, it is presently believed that there is a need and a potential commercial market for a medical product and method in accordance with the present invention.

### SUMMARY OF THE INVENTION

In essence, the present invention contemplates a medical product and a method for eliminating the symptoms of withdrawal from nicotine containing products such as cigarettes or other tobacco products. This medical product contains a mixture of 0.9% sodium chloride in water mixed with nicotine.

In the present invention, the Applicant's effort is to provide nicotine in the pattern and concentration to which those addicted to nicotine are accustomed.

In the preferred embodiment of the invention, medical products also include a cleaning solution of 1% to 3% ethyl alcohol, 1% citric acid for taste and 3% flavoring to mask the bitterness of nicotine.

A second embodiment of the invention relates to a method for eliminating the symptoms of withdrawal from a nicotine product by adding small concentrations of nicotine to the above solution. The amount of nicotine in a capsule containing 3 to 5 milliliters of mixture (solution) contains from about ⅛^{th} of a milligram to 3 milligrams of nicotine.

### DESCRIPTION OF THE PREFERRED EMBODIMENTS OF THE INVENTION

In a preferred embodiment of the present invention a nicotine/saline solution contains nicotine dissolved in a 0.9% sodium chloride solution with 1% to 3% of ethyl alcohol as a cleaning solution, 1% citric acid for taste and up to about 3% of a flavor.

A formulation method for a nicotine solution dissolved in 0.9% Sodium chloride solution with additives to be used with a micro micron mesh ultrasonic nebulizer technology to eliminate withdrawal symptoms in those addicted to nicotine. While Applicant's have referred to a micron micro mesh nebulizer, it should be recognized that any device that will produce particle sizes of 2-5 microns of saline/nicotine mixture or solution as defined herein may be substituted for the micron micro mesh nebulizer.

This application describes a formula for use with a micro micron mesh nebulizer in order to deliver solutions containing low concentrations of nicotine into the lungs in 2-5 micron particle sizes. The mixture solution is used to prevent withdrawal symptoms in those addicted to nicotine as a result of their use of tobacco products.

The necessity of eliminating withdrawal symptoms is important in a successful attempt to eliminate their use of tobacco products. It is the belief of the applicants that if equals are substituted for equals abstinence should occur. This embodiment describes the use of disposable capsules or cartridges to which are added small amounts of nicotine as well as 3% flavoring, 1% citric acid for taste, and 1-3% ethyl alcohol as a cleaning agent for the grid. These concentrations may vary. These solutions are contained in a capsule or cartridge made of plastic or silicone. The volume of these is 2-5 milliliters. These containers are perforated on both ends and then sealed with silicone tape.

When the cartridge is placed in the nebulizer cylinder anterior to the micro micron mesh grid; the tape is removed. The grid is activated by an on off switch and the solution is pulled through the device by the user drawing air through an entrainment port, the cylinder, the cartridge and the grid. The content of the nicotine solution varies in a declining manner in each cartridge from three milligrams, tapering by one eight of a milligram in a stepwise fashion. The lowest amount of nicotine in a cartridge is one eight of a milligram. The amount of nicotine described in this application may be modified depending on the severity of the nicotine addiction, but is not expected to exceed 3 milligrams per cartridge. To mask to taste of nicotine numerous flavors are available including citric acid, tobacco, apple, cherry, maple, menthol, just to name a few.

This allows an addicted individual to taper down the amount of nicotine needed to avoid withdrawal symptoms and hopefully quit the addiction completely and therefore eliminate the use of tobacco products.

The delivery into the lungs and therefore into the bloodstream of this solution and its contents is extremely efficient allowing 90% absorption rapidly in a pattern and concentration to which the nicotine addicted individual is accustomed. No heating of any liquid is involved. No refillable cartridge or capsule will be used thus avoiding dosage errors, spillage and contamination. The small doses of nicotine involved will avoid toxicity if a child or pet accidentally ingests a cartridge. No aerosol effluent is produced as the solution is housed in a cartridge and is drawn into the user's lungs by inhalation. The grid has multiple perforations of various sizes shapes, and number and is electrified by two 1.5 volt rechargeable NiCad batteries. This produces two micron sized particles. The sole source of flow through the device is the user's inspiratory effort and allows a rapid blood level of nicotine to be attained in a pattern to which nicotine addicted individuals are accustomed.

The pH of these solutions ranges from 5.5 to 7.4 for comfort and to decrease airway irritation and coughing. A pH of 7.4 is preferred as it enhances delivery of nicotine into the bloodstream.

While the invention has been described in connection with its preferred embodiments it should be recognized that changes and modifications may be made therein without departing from the scope of the appended claims.

1. A medical product for eliminating the symptoms of nicotine withdrawal from a nicotine containing product, said medical product comprising:
a nebulized solution including a mixture of a saline solution containing between ⅛^{th} and 3 milligrams of nicotine, 1 to 3% of ethyl alcohol as a cleaning solution, 1% citric acid for taste and 1% to 3% of a flavor; and
wherein said medical product has a pH of about 7.4; and
wherein said nebulized particle sizes of nicotine are two microns.

2. The medical product for eliminating the symptoms of withdrawal from a mixture containing product according to claim 1, in which the saline solution is a 0.9% sodium chloride solution in water and in which said flavors are selected from the group consisting of citrus, tobacco, apple, cherry, maple, menthol, hazelnut, peach, lemon, vanilla and chocolate.

3. The medical product for eliminating the symptoms of withdrawal from a mixture containing product according to claim 2, which includes a capsule containing 3 to 5 ml of said mixture disposed in said capsule and wherein each capsule contains between about ⅛ milligram and 3 milligrams of nicotine.

4. The medical product for eliminating the symptoms of withdrawal from a mixture containing product according to claim 1, in which each capsule contains about ¼ milligram of nicotine.

5. The medical product for eliminating the symptoms of withdrawal from a mixture containing product according to claim 1, in which each capsule contains about ⅝ milligram of nicotine.

6. The medical product for eliminating the symptoms of withdrawal from a mixture containing product according to claim 1, in which each capsule contains about ¾ milligram of nicotine.

7. The medical product for eliminating the symptoms of withdrawal from a mixture containing product according to claim 1, in which each capsule contains about ⅞ milligram of nicotine.

8. The medical product for eliminating the symptoms of withdrawal from a mixture containing product according to claim 1, in which each capsule contains about 1 milligram of nicotine.

9. The medical product for eliminating the symptoms of withdrawal from a mixture containing product according to claim 1, in which each capsule contains about 1¼ milligrams of nicotine.

10. The medical product for eliminating the symptoms of withdrawal from a mixture containing product according to claim 1, in which each capsule contains about 1⅜ milligrams of nicotine.

11. The medical product for eliminating the symptoms of withdrawal from a mixture containing product according to claim 1, in which each capsule contains about 1½ milligrams of nicotine.

12. The medical product for eliminating the symptoms of withdrawal from a mixture containing product according to claim 1, in which each capsule contains about 1⅝ milligrams of nicotine.

13. The medical product for eliminating the symptoms of withdrawal from a mixture containing product according to claim 1, in which each capsule contains about 1¾ milligrams of nicotine.

14. The medical product for eliminating the symptoms of withdrawal from a mixture containing product according to claim 1, in which each capsule contains about 2 milligrams of nicotine.

15. The medical product for eliminating the symptoms of withdrawal from a mixture containing product according to claim 1, in which each capsule contains about 2¼ milligrams of nicotine.

16. The medical product for eliminating the symptoms of withdrawal from a mixture containing product according to claim 1, in which each capsule contains about 2½ milligrams of nicotine.

17. The medical product for eliminating the symptoms of withdrawal from a mixture containing product according to claim 1, in which each capsule contains about 2¾ milligrams of nicotine.

18. The medical product for eliminating the symptoms of withdrawal from a mixture containing product according to claim 1, in which each capsule contains about 3 milligrams of nicotine.

19. A method for eliminating the symptoms of withdrawal from a nicotine containing product, said method comprising:
a. providing a saline/nicotine solution containing from about one (1) to about three (3) milligrams of nicotine saline solution;
b. nebulizing said saline/nicotine solution with an electrically charged micro mesh grid to produce a nicotine particle size of between about 2 and about 5 microns;
c. inhaling the 2 to 5 micron particles of a nebulized nicotine concentration for a first preselected period of time;
d. providing a second reduced concentration of a saline/nicotine solution;
e. nebulizing said second reduced concentration with a micro mesh grid to produce nicotine particles of about 2 microns; and
f. inhaling the 2 micron particles from step e for a second period of time.

20. The method for eliminating the symptoms of withdrawal from inhaling a nicotine containing product according to claim 19, in which the micro mesh grid is electrified to vibrate said grid in step b and e by two 1.5 volt nickel cadmium batteries;
in which the step e and f are each repeated until the individual has eliminated the symptoms of withdrawal.
